(19) United States
(12) Patent Application Publication
(10) Pub. No.: US 2026/0083305 A1
(43) Pub. Date: Mar. 26, 2026

(54) ASPIRATION TUBING CLIP

(71) Applicant: BOSTON SCIENTIFIC SCIMED, INC., Maple Grove, MN (US)

(72) Inventors: Cassandra Anne Hailson, Marlborough, MA (US); Troy Velazquez, Salem, NH (US); Jessica Williams, Marlborough, MA (US)

(73) Assignee: BOSTON SCIENTIFIC SCIMED, INC., Maple Grove, MN (US)

(21) Appl. No.: 19/337,425

(22) Filed: Sep. 23, 2025

Related U.S. Application Data
(60) Provisional application No. 63/698,293, filed on Sep. 24, 2024.

(51) Int. Cl. A61B 1/015; A61B 1/307; A61M 39/28

(52) U.S. Cl. CPC A61B 1/015; A61B 1/307; A61M 39/281; A61M 2205/3344

(57) ABSTRACT

A fluid system including a fluid management system, a fluid cassette, a suction source, a suction tubing, and an actuatable clamp. The fluid management system may include a fluid management console which may include a housing, a controller housed within the housing, an inflow pump disposed within the housing and having a pump motor, and a user input interface. The fluid cassette may be configured to be received within a receptacle of the housing of the fluid management console to provide a flow of fluid to a medical device. The suction tubing may be configured to provide a flow fluid from the medical device and the actuatable clamp may be positioned about an outer surface of the suction tubing.

### CROSS-REFERENCE TO RELATED APPLICATIONS

The application claims the benefit of U.S. Provisional Patent Application Ser. No. 63/698,293, filed on Sep. 24, 2024, the disclosure of which is incorporated herein by reference.

### TECHNICAL FIELD

The disclosure is directed to a fluid management system. More particularly, the disclosure is directed to methods and systems for flow control in a fluid management system.

### BACKGROUND

Flexible ureteroscopy (fURS), gynecology, and other endoscopic procedures require the circulation of fluid for several reasons. Surgeons today deliver the fluid in various ways such as, for example, by hanging a fluid bag and using gravity to deliver the fluid, filling a syringe and manually injecting the fluid or using a peristaltic pump to deliver fluid from a reservoir at a fixed pressure or flowrate via a fluid management system. Fluid management systems may be improved with the addition of suction. Of the known medical devices, systems, and methods, each has certain advantages and disadvantages. There is an ongoing need to provide alternative medical devices and fluid delivery systems.

### BRIEF SUMMARY

This disclosure provides design, material, manufacturing method, and use alternatives for components of a fluid management system.

In a first example, a fluid system may comprise a fluid management system including a fluid management console having a housing, a controller housed within the housing, an inflow pump disposed within the housing and having a pump motor, and a user input interface. The fluid system may further include a fluid cassette configured to be received within a receptacle of the housing of the fluid management console, the fluid cassette configured to provide a flow of fluid to a medical device, a suction source, a suction tubing operatively coupled with the suction source, the suction tubing configured to provide a flow of fluid from the medical device, and an actuatable clamp positioned about an outer surface of the suction tubing.

Alternatively or additionally to any of the examples above, in another example, the actuatable clamp may be configured to selectively apply a compressing force to the suction tubing.

Alternatively or additionally to any of the examples above, in another example, the actuatable clamp may be movable between a fully open configuration and a fully closed configuration.

Alternatively or additionally to any of the examples above, in another example, the actuatable clamp may be positionable in a configuration between the fully open configuration and the fully closed configuration.

Alternatively or additionally to any of the examples above, in another example, when the actuatable clamp is in the fully open configuration the flow of fluid from the medical device may be at a maximum.

Alternatively or additionally to any of the examples above, in another example, when the actuatable clamp is in the fully closed configuration the flow of fluid from the medical device may be at a minimum.

Alternatively or additionally to any of the examples above, in another example, the actuatable clamp may be communicatively coupled to the controller of the fluid management console.

Alternatively or additionally to any of the examples above, in another example, the actuatable clamp may comprise at least one pressure sensor configured to measure a pressure within the suction tubing.

Alternatively or additionally to any of the examples above, in another example, the actuatable clamp may comprise an elongate tubular body having a lumen extending therethrough.

Alternatively or additionally to any of the examples above, in another example, the actuatable clamp may comprise a first body member hingedly coupled to a second body member.

Alternatively or additionally to any of the examples above, in another example, the actuatable clamp may comprise an actuatable member. The actuatable member may be configured to be displaced into a lumen of the actuatable clamp.

Alternatively or additionally to any of the examples above, in another example, the actuatable member is coupled to an actuation member.

Alternatively or additionally to any of the examples above, in another example, the actuatable clamp may be configured to selectively reduce a diameter of the suction tubing.

Alternatively or additionally to any of the examples above, in another example, the actuatable clamp may be configured to apply a compressive force around less than an entirety of a circumference of the suction tubing.

Alternatively or additionally to any of the examples above, in another example, the actuatable clamp may be configured to apply a compressive force around an entirety of a circumference of the suction tubing.

In another example, a fluid system may comprise a fluid management system including a fluid management console including a housing, a controller housed within the housing, an inflow pump disposed within the housing and having a pump motor, and a user input interface. The fluid system may further comprise a fluid cassette configured to be received within a receptacle of the housing of the fluid management console, the fluid cassette configured to provide a flow of fluid to a medical device, a suction source, a suction tubing operatively coupled with the suction source, the suction tubing configured to provide a flow of fluid from the medical device, and an actuatable clamp removably positioned about an outer surface of the suction tubing and in electronic communication with the controller of the fluid management console, the actuatable clamp configured to selectively change a volume of the flow of fluid through the suction tubing.

Alternatively or additionally to any of the examples above, in another example, the actuatable clamp may be movable between a fully open configuration and a fully closed configuration.

Alternatively or additionally to any of the examples above, in another example, the actuatable clamp may be positionable in a configuration anywhere between and including the fully open configuration and the fully closed configuration.

In another example, actuatable clamp configured to selectively control a flow of fluid from a medical device may comprise an elongate tubular body extending from a proximal end to a distal end and having a lumen extending therethrough. The elongate tubular body may comprise a first body member, a second body member hingedly coupled to the first body member, and an actuatable member configured to be selectively displaced into the lumen of the elongate tubular body. The first body member and the second body member may be movable between a closed configuration and an open configuration configured to provide a lateral opening to the lumen of the elongate tubular body.

Alternatively or additionally to any of the examples above, in another example, the actuatable clamp may further comprise at least one pressure sensor disposed adjacent to the lumen of the elongate tubular body.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set;

FIG. 3 is a schematic view of an illustrative medical device that may be used in conjunction with the fluid management system of FIGS. 1-2;

FIG. 4 is a schematic view of an illustrative endoscopic system including a fluid management system, an endoscope, an aspiration introducer sheath, and a waste management system;

FIG. 5 is a perspective view of an illustrative actuatable clamp;

FIG. 6A is a schematic cross-sectional view of the illustrative actuatable clamp of FIG. 5 disposed over an aspiration tubing with the actuatable clamp in a first configuration; and

FIG. 6B is a schematic cross-sectional view of the illustrative actuatable clamp of FIG. 5 disposed over an aspiration tubing with the actuatable clamp in a second configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, but not limited to, a LithoVue™ Elite endoscope device using pressure and/or temperature data from the endoscope or other endoscopic device. The fluid management system may provide fluid to the body. In some cases, the introduction of fluid into the body from the fluid management system may be controlled by limiting the intraluminal pressure (ILP) as measured at a distal end of the endoscope device. The fluid management system may be configured to reduce fluid flow as an ILP limit is approached. However, the fluid management system may not have the ability to provide controlled aspiration of fluid from the body. Fluid removal may be achieved through a suction device and/or system separate from the fluid management system, the control of which may be separate from the control of the fluid management system. Thus, if an ILP limit is exceeded the fluid management system relies on the external outflow control (passive or active) for the intraluminal pressure to be relieved or lowered. The present disclosure is directed towards systems and methods for controlling fluid flow through the suction system to improve performance of the fluid management system. While the present disclosure is described with respect to urological procedures, the systems and methods described herein may be used in other anatomies, as desired.

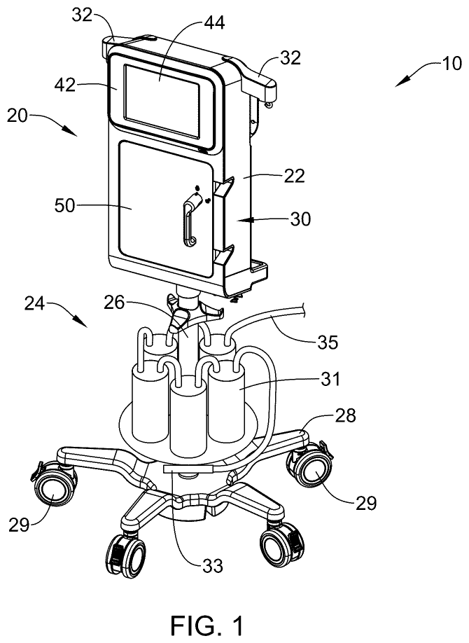

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the LithoVue™ Elite endoscope, or other endoscope. In some instances, the endoscope may include a temperature sensor to provide intraluminal temperature feedback to the fluid management system 10, a pressure sensor to provide intraluminal pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. The wheeled cart 24 may include a pole 26 extending upward from a base 28 including a plurality of wheels 29 (e.g., caster wheels). In other instances, the console 20 may be provided with another form of cart, configured to be positioned on a flat surface, mounted to a wall, etc.

The fluid management system 10 may also include one or more user input interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The user input interface, e.g., touch screen interface 42, allows the user to input/adjust various functions of the fluid management system 10 such as, for example flowrate, pressure, and/or temperature. The user may also configure parameters and alarms, information to be displayed, and the procedure mode. The user input interface, e.g., touch screen interface 42, allows the user to add, change, and/or discontinue the use of various modular systems within the fluid management system 10. The user input interface, e.g., touch screen interface 42, may also be used to change the fluid management system 10 between automatic and manual modes for various procedures. It is contemplated that other systems configured to receive user input may be used in place of or in addition to the touch screen interface 42, such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flowrate and/or fluid pressure display. In some embodiments, operating parameters may be adjusted by touching a corresponding portion of the touch screen interface 42. The touch screen interface 42 may also display visual alerts and/or audio alarms if parameters (e.g., flowrate, temperature, etc.) are above or below predetermined and/or user-adjustable thresholds and/or ranges. In some embodiments, the fluid management system 10 may also include further user interface components such as an optional foot pedal, a fluid warmer user interface, a fluid control interface, or other devices to manually control various modular systems. For example, an optional foot pedal may be used to manually control flowrate. Some illustrative display screens 44 and other user interface components are described in commonly assigned U.S. Patent Application Publication No. 2018/0361055, titled AUTOMATED FLUID MANAGEMENT SYSTEM, the entire disclosure of which is hereby incorporated by reference.

The user input interface, e.g., touch screen interface 42, may be operatively connected to or a part of the controller 30. The controller 30 may be a CPU, including a computer, tablet computer, or other processing device. The controller 30 may be operatively connected to one or more system components such as, for example, an inflow pump, an outflow or vacuum pump, a fluid warming system, an aspiration tubing clamp, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flowrate or the fluid pressure of fluid provided by the fluid management system 10 at any given time may be displayed on the display screen 44 to allow the operating room (OR) visibility for any changes. If the OR personnel notice a change in fluid flowrate or fluid pressure that is either too high or too low, the user may manually adjust the fluid flowrate or the fluid pressure back to a preferred level. The fluid management system 10 may also monitor and automatically adjust the fluid flowrate (e.g., inflow or outflow) or the fluid pressure based on previously set parameters.

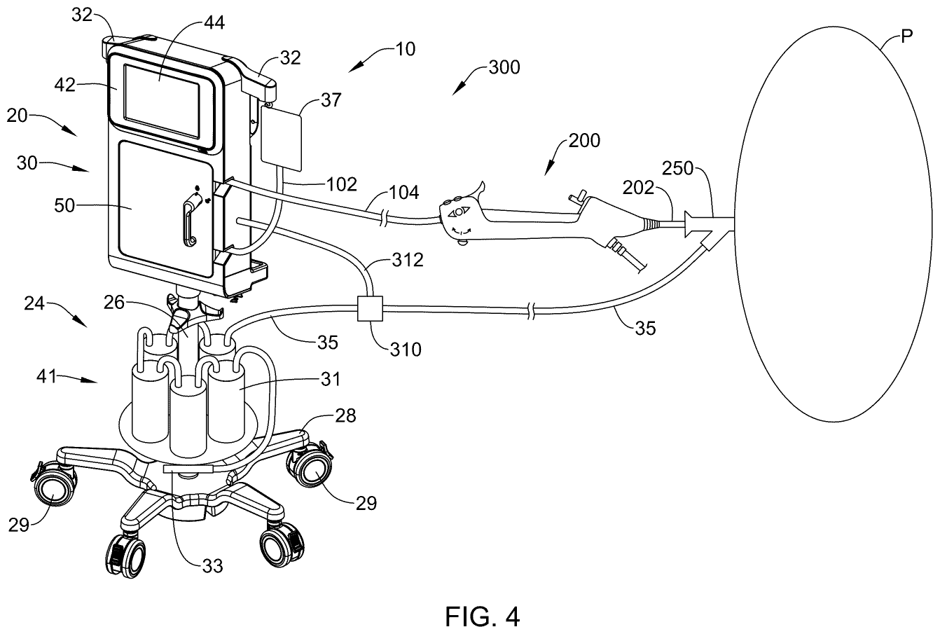

An illustrative fluid management unit may include one or more fluid container supports, such as fluid supply source hangers 32, each of which may support a fluid supply source (e.g., fluid bag) 37 (see, for example, FIG. 4). In some embodiments, placement and/or weight of the fluid supply source(s) 37 hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) 37 such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources 37 may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more optional collection containers 31, for collecting waste fluid during a medical procedure. The collection containers 31 (e.g., canisters) may be in fluid communication with an optional vacuum or outflow pump 33 to provide suction for drawing fluid into the collection containers 31. The vacuum pump 33 may be operatively and/or electronically connected to the controller 30. In some embodiments, the vacuum pump 33 may be disposed within the fluid management system 10. Other configurations are also contemplated. In some embodiments, the collection container(s) 31 may be operatively coupled to a collection load cell to detect placement and/or weight of fluid in the collection container(s) to contribute to a fluid deficit calculation. The collection container(s) 31 may be fluidly connected to the medical device or a different medical device (e.g., aspiration sheath) via a flexible aspiration tubing 35. The aspiration tubing 35 may be fluidly connected to the medical device in a number of different configurations. For example, the aspiration tubing 35 may be fluidly connected to an aspiration port on the medical device, to an access or aspiration sheath disposed over the medical device, to a tool configured to be inserted into the working channel of the medical device, etc.

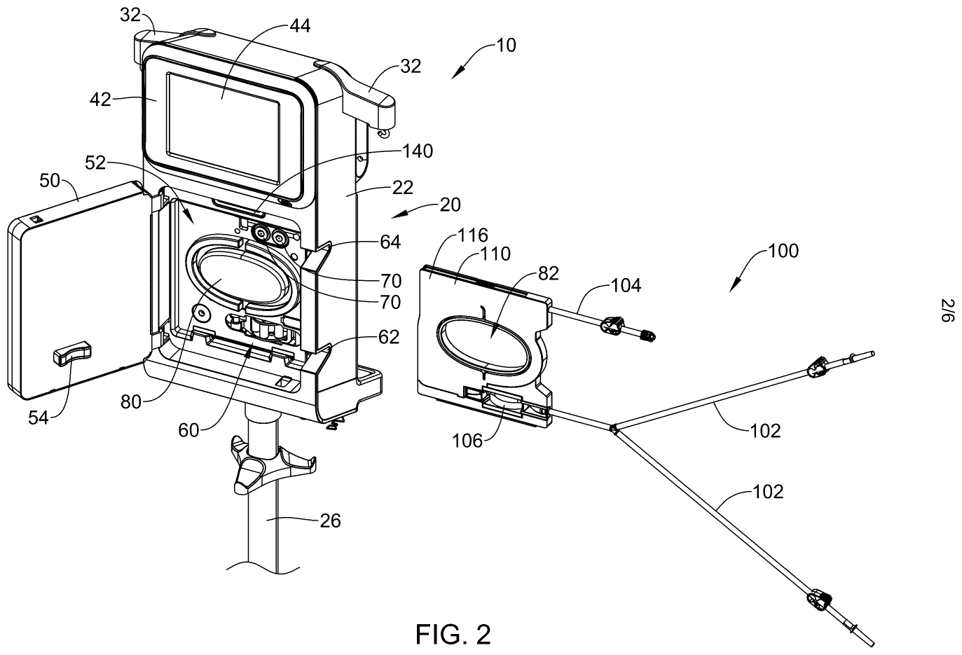

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flowrate or a target fluid pressure. The inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target or predetermined fluid flowrate or target fluid pressure based on a set of system operating parameters. In some embodiments, the controller 30 may be configured to control the inflow pump 60 to maintain a desired fluid pressure at the treatment site or a predetermined flowrate based on a set of system operating parameters.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flowrate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flowrate, fluid pressure, and/or other parameters. Once the user has selected the manual mode on, for example, the touch screen interface 42, the user may the adjust fluid flowrate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike, or other connector, for connecting to the fluid supply source(s). The single use tubing set 100 may also include outflow tubing 104 providing a fluid outflow from the interior of the cassette 110 to a medical device connected thereto. The single use tubing set 100, including the fluid cassette 110, the inflow tubing 102, and the outflow tubing 104, may be disposable and provided sterile and ready to use.

When the fluid cassette 110 is installed in the receptacle 52 and the door 50 is closed, the inflow tubing 102 may pass through a channel 62 extending through a wall of the housing 22 of the console 20 to an exterior of the console 20. Likewise, when the fluid cassette 110 is installed in the receptacle 52 and the door 50 is closed, the outflow tubing 104 may pass through a channel 64 extending through a wall of the housing 22 of the console to an exterior of the console 20. The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flowrates required in the specific application of the fluid management system 10. Some illustrative fluid warming systems are described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

The console 20 may further include one or more additional sensors, such as a pressure sensor and/or a bubble sensor. For instance, the console 20 may include a pressure sensor 70, illustrated as a pair of pressure sensors, configured to monitor a system pressure (i.e., pump pressure) of fluid exiting the cassette 110 and flowing through the outflow tubing 104 to a surgical site. The fluid cassette 110 may include a corresponding pressure sensor interface (not explicitly shown), such as a flexible membrane, that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44. Additional features of the cassette 110 of the fluid tubing set 100 are described in commonly assigned U.S. Patent Application No. 63/640,089, titled DEVICES, SYSTEMS, AND METHODS FOR FLOW COMPENSATION IN A FLUID MANAGEMENT SYSTEM, the entire disclosure of which is hereby incorporated by reference.

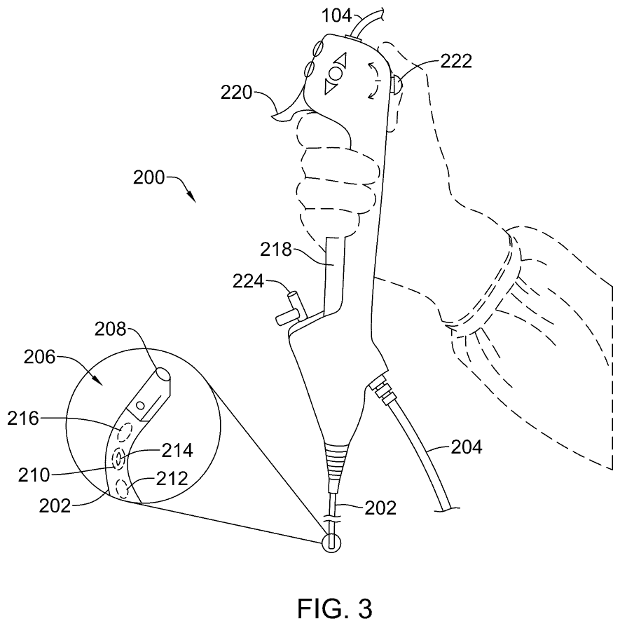

FIG. 3 illustrates aspects of a medical device 200 that may be used in conjunction with the fluid management system 10. In the illustrated embodiments, the medical device 200 may be a ureteroscope such as a LithoVue™ Elite endoscope, another intraluminal pressure sensing endoscope, or other endoscope. However, other medical devices, such as another endoscope, may be used in addition to or in place of a ureteroscope. The medical device 200 may be configured to deliver fluid from the fluid management system 10 to the treatment site via an elongate shaft 202 configured to access the treatment site within the patient. In some embodiments, the inflow pump 60 may be in fluid communication with the elongate shaft 202. The elongate shaft 202 may include one or more working lumens for receiving a flow of fluid or other medical devices therethrough. The medical device 200 is connected to the fluid management system 10 via one or more supply line(s) 104 (e.g., a tube), as shown in FIG. 2 for example.

In some embodiments, the medical device 200 may be in electronic communication with a workstation (not explicitly shown) via a wired connection 204. The workstation may be in wired or wireless communication with the controller 30 of the fluid management system 10. In some embodiments, the workstation may be a multi-use component (e.g., used for more than one procedure) while the medical device 200 may be a single use device, although this is not required. In some embodiments, the workstation may be omitted and the medical device 200 may be electronically coupled directly to the controller 30 of the fluid management system 10.

As shown in FIG. 3, the medical device 200 may include one or more sensors proximate a distal end 206 of the elongate shaft 202. For example, the medical device 200 may include a pressure sensor 208 at a distal tip of the elongate shaft 202 to measure intraluminal pressure within the treatment site. The medical device 200 may also include other sensors such as, for example, a temperature sensor 210, a Fiber Bragg grating optical fiber 212 to detect stresses, and/or an antenna or electromagnetic sensor 214 (e.g., a position sensor). In an illustrative embodiment, the distal end 206 of the medical device 200 may also include at least one camera 216 to provide a visual feed to the user on the display screen of the workstation. In another embodiment, the medical device 200 may include two cameras 216 having different communications requirements or protocols so that different information may be relayed to the user by each camera 216. When so provided, the user may switch back and forth between cameras 216 at will through the touch screen interface 42 and/or the workstation. While not explicitly shown, the elongate shaft 202 may include one or more working lumens for receiving the fluid and/or other medical devices.

The medical device 200 includes a handle 218 coupled to a proximal end of the elongate shaft 202. The handle 218 may have a fluid flow on/off switch 220, which allows the user to control when fluid is flowing through the medical device 200 and into the treatment site. The handle 218 may further include other buttons 222 that perform other various functions. For example, in some embodiments, the handle 218 may include buttons to control the temperature of the fluid. It will be understood that while the exemplary embodiment describes a ureteroscope, the features detailed above may also be directly integrated into a cystoscope, an endoscope, a hysteroscope, or virtually any device with an image capability. In some embodiments, the medical device 200 may also include a drainage port 224 which may be connected to a drainage system such as the vacuum pump 33 and the collection containers 31. Some illustrative drainage systems are described in commonly assigned U.S. Patent Application Publication No. 2018/0361055, titled AUTOMATED FLUID MANAGEMENT SYSTEM, the disclosure of which is hereby incorporated by reference.

As described herein, it may be desirable to control a flow of fluid out of the patient through aspiration. Typically, aspiration may be performed using sources of suction separate from the fluid management system 10, such as, but not limited to, benchtop suction vacuum pumps, wall suction, waste management systems, or the like. FIG. 4 is a schematic view of an illustrative endoscopic system 300 including a fluid management system 10, an endoscope 200, an aspiration introducer sheath 250, and a waste management system 41. The aspiration introducer sheath 250 may be introduced into the patient P with the elongate shaft 202 of the endoscope 200 extending through a lumen of the aspiration introducer sheath 250 and into the patient P. As described herein, the fluid inflow tubing 104 is fluidly coupled to the endoscope 200 to provide a flow of fluid into the patient P. The aspiration introducer sheath 250 may be connected to any suction source. For example, the present system may use a suction source that is controlled separately from the fluid management system 10. However, the present system may not require the suction to be adjusted via the separate system.

The aspiration tubing 35 extends from a proximal end fluidly coupled to a collection container 31 of the waste management system 41 to a distal end fluidly coupled to the aspiration introducer sheath 250. Activating the vacuum pump or outflow pump 33 pulls a vacuum on the collection container(s) which in turn pulls fluid from the patient P through a lumen of the aspiration tubing 35. In some cases, the outflow pump 33 may be omitted entirely. Alternative suction sources (e.g., benchtop suction vacuum pumps, wall suction, waste management systems, etc.) may be used in place of an outflow pump. Further, passive suction may also be used. The aspiration tubing 35 may include an actuatable valve or clamp 310 positioned about an outer surface thereof. The actuatable clamp 310 may be electronically and communicably coupled to the fluid management system 10 through a wire or cable 312. In some cases, the cable 312 may provide power and/or communication capabilities to the actuatable clamp 310. Generally, the actuatable clamp 310 may include an actuatable member configured to apply a compressive force on an outer surface of the aspiration tubing 35 to reduce an inner diameter of the aspiration tubing 35 to control a volume of fluid flowing therethrough. For example, reducing an inner diameter of the lumen of the aspiration tubing 35 reduces the volume of fluid flowing through the aspiration tubing 35. The controller 30 of the fluid management system 10 may be communicatively coupled to the actuatable clamp 310 via the cable 312. This may allow the fluid management system 10 to send control commands to the actuatable clamp 310 to actively control the volume of fluid being aspirated or removed from the patient P. It is contemplated that when the controller 30 of the fluid management system 10 controls the aspiration flowrate and/or volume, the controller 30 may follow the ILP maximum pressure or limit without sacrificing flush and/or flow responsiveness. As noted above, in the absence of control over the aspiration flowrate and/or volume, when the ILP limit is approached, the fluid management system 10 relies on the suction system (separately controlled from the fluid management system 10) or passive outflow to reduce the pressure within the patient P. Further, when the controller 30 of the fluid management system 10 controls the aspiration flowrate and/or volume, the user may control all irrigation function via the graphical user interface (GUI) or touch screen interface 42 of the fluid management system 10. Thus, a user is not required to access a separate control system (which may be spaced from the fluid management system 10) to adjust the aspiration settings. As the actuatable clamp 310 is positioned about an outer surface of the aspiration tubing 35, the actuatable clamp 310 may not require sterilization and may be reused across multiple procedures. Said differently, the inner and outer surfaces of the actuatable clamp 310 are free from contact with bodily fluids or contaminants that may require sterilization of the actuatable clamp 310.

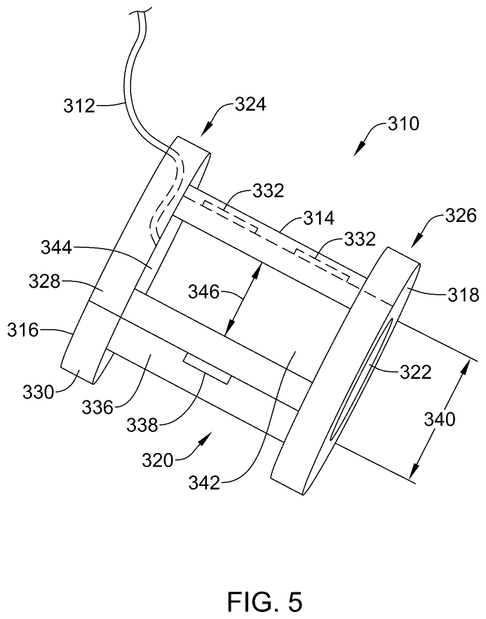

FIG. 5 is a perspective view of the illustrative actuatable clamp 310. In some instances, the actuatable clamp 310 may be formed from an elongated tubular body 314. While the actuatable clamp 310 is described as generally tubular, it is contemplated that the actuatable clamp 310 may take any cross-sectional shape desired. The actuatable clamp 310 may have a first, or proximal end 316, a second, or distal end 318, and an intermediate region 320 disposed between the first end 316 and the second end 318. The actuatable clamp 310 may include a lumen 322 extending from a first opening adjacent the first end 316 to a second opening adjacent to the second end 318 to allow for the passage of dust, stone fragments, fluids, etc.

In some instances, the actuatable clamp 310 may include a first end region 324 proximate the proximal end 316 and a second end region 326 proximate the second end 318. In some embodiments, the first end region 324 and the second end region 326 may have enlarged diameters relative to the intermediate region 320. However, this is not required. In some examples, the actuatable clamp 310 may have a substantially uniform diameter along the length thereof. When so provided, the enlarged end regions 324, 326 may provide a grasping region for manipulating the actuatable clamp 310. It is contemplated that a transition from the cross-sectional area of the intermediate region 320 to the enlarged end regions 324, 326 may be gradual, sloped, or occur in an abrupt stepwise manner, as desired.

The actuatable clamp 310 may include a first body member 328 and a second body member 330. The first body member 328 and the second body member 330 may be of similar sizes (e.g., each forming about half of the actuatable clamp 310). However, this is not required. One of the first or second body members 328, 330 may be larger than the other. The first and second body members 328, 330 may be pivotably or hingedly coupled to one another via one or more hinges 332 such that the first body member 328 and the second body member 330 may be moved relative to one another. For example, pivoting the first body member 328 relative to the second body member 330 may allow a first portion 334 of the first body member 328 to move away from a first portion 336 of the second body member 330 creating an opening extending generally parallel to a longitudinal axis of the actuatable clamp 310. In another example, the first body member 328 and the second body member 330 may be connected by a flexible region. When the first body member 328 is moved away from the second body member 330, the actuatable clamp 310 may be positioned over the aspiration tubing 35 in non-parallel direction relative to a longitudinal axis of the aspiration tubing 35. Said differently, the actuation of the first body member 328 and/or the second body member 330 may allow the actuatable clamp 310 to be positioned over the aspiration tubing 35 without sliding the actuatable clamp 310 along a length of the aspiration tubing 35 to the desired location. A releasable clasp 338, or other releasable locking mechanism may releasably secure the first body member 328 to the second body member 330 to maintain the actuatable clamp 310 in a closed configuration.

The lumen 322 of the actuatable clamp 310 may have diameter 340 that is approximately the same as or larger than the outer diameter of the aspiration tubing 35. This may allow the actuatable clamp 310 to be secured about the outer surface of the aspiration tubing 35 without applying a compressive force to the wall of the aspiration tubing 35. In some cases, the diameter 340 of the actuatable clamp 310 may be sized to provide a friction fit between the wall of the lumen 322 and the outer surface of the aspiration tubing 35. This may help prevent the actuatable clamp 310 from sliding or moving along a length of the aspiration tubing 35. However, this is not required.

The actuatable clamp 310 may further include an actuatable member 342, such as, but not limited to, an actuatable flap. In other examples, the actuatable member 342 may be a ridge or protrusion formed in the first body member 328 similar in form and function to a medical tubing clamp. The actuatable member 342 may be configured to be moved into the lumen 322 of the actuatable clamp 310 to selectively compress the wall of the aspiration tubing 35 to reduce an inner diameter of the aspiration tubing 35. The actuatable member 342 may take a number of different forms. In some examples, the actuatable member 342 may be formed from a portion of the wall of the intermediate region 320 of the actuatable clamp 310. In other examples, the actuatable member 342 may be a separate component disposed within the lumen 322 of the actuatable clamp 310 and movably coupled thereto and electronically coupled to the cable 312. The actuatable member 342 may be coupled to an actuation member 344. For example, the actuation member 344 may be a rotating or pivoting member such as, but not limited to, a hinge. In other examples, the actuation member 344 may be configured to exert a biasing force on an external surface of the actuatable member 342, such as, but not limited to, a pinching force. The actuation member 344 may be configured to actuate or move the actuatable member 342 in response to a signal received from the controller 30 of the fluid management system 10 via the cable 312 which is electronically coupled to the actuatable member 342. In some embodiments, the actuatable member 342 may receive wireless communication signals from the controller 30, such as, but not limited to, one or more wireless communication protocols such as cellular communication, ZigBee, REDLINK™, Bluetooth, WiFi, 5G, LiFi, a SIM card, dedicated short range communication (DSRC), EnOcean, and/or any other suitable common or proprietary wireless protocol, as desired. It is further contemplated that the actuatable clamp 310 may include a power source such as, but not limited to, a battery or rechargeable battery.

The actuation member 344 may have an arc length 346 that is less than a circumference of the intermediate region 320 of the actuatable clamp 310. In some cases, the arc length 346 of the actuatable member 342 may be less than half the circumference of the intermediate region 320. However, this is not required. In some configurations, the actuatable member 342 may be configured to apply a circumferential force to the outer surface of the aspiration tubing 35.

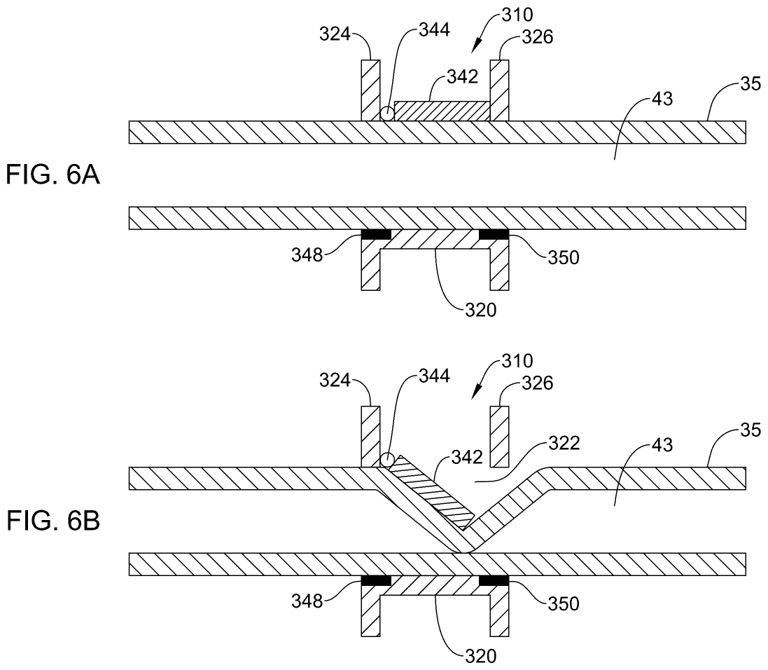

FIG. 6A is a schematic cross-sectional view of the illustrative actuatable clamp 310 disposed over an aspiration tubing 35 with the actuatable clamp 310 in a first configuration and FIG. 6B is a schematic cross-sectional view of the illustrative actuatable clamp 310 disposed over an aspiration tubing 35 with the actuatable clamp 310 in a second configuration. In the first configuration (FIG. 6A), the actuatable member 342 is in a fully open position which does not exert a biasing force on the wall of the aspiration tubing 35. Thus, the lumen 43 of the aspiration tubing 35 has a maximum diameter. This may allow for a maximum volume of the fluid to be withdrawn from the patient P for a given speed of the vacuum pump 33 (or other suction source). When the controller 30 determines a reduction of the flowrate or volume of fluid exiting the patient P is needed or desired, the controller 30 may send a control command to the actuation member 344. In response to the control command, the actuation member 344 actuates or moves the actuatable member 342 radially inwards into the lumen 322 of the actuatable clamp 310 towards a closed configuration. The radially inward movement of the actuatable member 342 may apply a radially inwards compressive biasing force to a wall of the aspiration tubing 35. This may cause the inner diameter of the aspiration tubing 35 to reduce. In some cases, the compression force exerted by the actuatable member 342 may be correlated to the size and/or material of the aspiration tubing 35 to avoid or minimize damage to the aspiration tubing 35. The diameter of the aspiration tubing 35 may be input at the touch screen interface 42 or sensed by the actuatable clamp 310, as desired. FIG. 6B illustrates the actuatable member 342 in a minimum flow configuration in which the opposing sides of the wall of the aspiration tubing 35 are brought into contact with one another to completely or substantially block a flow of fluid through the lumen 43 of the aspiration tubing 35. The actuation member 344 may be configured to position the actuatable member 342 at any position between the maximum flow configuration and the minimum flow configuration such that the flow of fluid through the lumen 43 of the aspiration tubing 35 may be incrementally or infinitely adjusted.

In some examples, the controller 30 may be configured to automatically control a position of the actuatable member 342 based on the measured ILP (as measured at pressure sensor 208), the setpoint ILP, a measured fluid inflow flowrate (e.g., fluid flow through fluid management system 104), a setpoint fluid inflow flowrate (e.g., setpoint of fluid flow through fluid management system 104), a flush mode (e.g., a temporary increase in fluid inflow through the fluid management system 104), a distention mode (e.g., a temporary increase in the setpoint ILP), or the like. Alternatively, or additionally, the controller 30 may be configured to send a control command to the actuation member 344 in response to a user input at the touch screen interface 42. The user input may be a direct command to increase or decrease a flow of outflow fluid through the aspiration tubing 35 or may be a command to change a different operational setpoint (e.g., ILP, inflow flowrate, or the like). It is contemplated that the maximum suction (e.g., fluid outflow) may be determined by the settings at the suction source. For example, in the illustrated embodiment, the maximum suction with the actuatable clamp 310 is limited by the maximum suction of the vacuum pump 33 of the waste management system 41. Said differently, the user may not increase the flowrate of fluid through the aspiration tubing 35 above the maximum flowrate provided by the settings and/or capacity of the vacuum pump 33 (or other vacuum source). It is further contemplated that the actuatable member 342 may be actuated to simulate a higher input pressure by providing a negative pressure at the outflow. For example, the pressure differential between the inflow and the outflow may be equivalent to a higher inflow pressure in the absence of suction.

In one illustrative example, the actuatable member 342 may be actuated to a more open configuration (e.g., exert less compressive force on the aspiration tubing 35) to decrease an ILP. The actuatable member 342 may be actuated to a more closed configuration (e.g., exert more compressive force on the aspiration tubing 35) to increase an ILP. It is contemplated that the user may set the source suction (e.g., vacuum pump 33) to a maximum value according to user preferences or safety guidelines. The user may then set a position of the actuatable member 342 based on a desired flowrate or pressure relative to the maximum value. For example, if the user wants the maximum suction, the user may select 100% open at the touch screen interface 42. If the user wants no suction or zero outflow, the user may select 0% open at the touch screen interface 42. It is contemplated that the controller 30 may be configured to automatically determine how “open” the actuatable member 342 should be to achieve the desired flowrate. In some cases, the position of the actuatable member 342 may be correlated to a current and/or voltage. For example, an increase or spike in voltage to the actuation member 344 may indicate the actuatable member 342 is moving towards or is at the fully closed position.

In some embodiments, the actuatable clamp 310 may include one or more optional pressure sensors 348, 350. For example, the actuatable clamp 310 may include an optional proximal pressure sensor 348 adjacent to the first end region 324. When the actuatable clamp 310 is disposed over the aspiration tubing 35, the proximal pressure sensor 348 may be positioned on the side of the actuatable clamp 310 closer to the suction source (e.g., vacuum pump 33). The actuatable clamp 310 may include visual indicia or markings to help the user orientate the actuatable clamp 310 relative to the suction source and/or relative to the patient P. The proximal pressure sensor 348 may provide the user with a pressure value of the suction delivered by the pressure source. This may allow the user to have a numerical representation of the suction pressure setpoint (e.g., as set at the suction source) which may not be available from the suction source (for example, when the suction source includes analog controls). The actuatable clamp 310 may include an optional distal pressure sensor 350 adjacent to the second end region 326. When the actuatable clamp 310 is disposed over the aspiration tubing 35, the distal pressure sensor 350 may be positioned on the side of the actuatable clamp 310 closer to the patient P. The actuatable clamp 310 may include visual indicia or markings to help the user orientate the actuatable clamp 310 relative to the suction source and/or relative to the patient P. The distal pressure sensor 350 may provide a measure of the suction pressure being transferred to the aspirating device (e.g., the aspirating introducer sheath 250 in the illustrated example). This may allow the user to select a suction setting based on pressure (mm/Hg) as opposed to a percent open/closed. It is contemplated that the actuatable member 342 may include only the proximal pressure sensor 348, only the distal pressure sensor 350, both the proximal and distal pressure sensors 348, 350, or no pressure sensors, as desired. It is contemplated that when both the proximal pressure sensor 348 and the distal pressure sensor 350 are provided, the ILP may be estimated based on the pressure differential.

The actuatable clamp 310 may allow for control of the aspiration within the endoscopic system 300 without the addition of another pump on the fluid management system 10 or through the use of two separate control systems. This may save space, reduce power consumption, reduce noise, reduce design complexity of the fluid management system 10, increase the performance of the fluid management system 10 without increased capital costs. It is further contemplated that the actuatable clamp 310 may have built-in clog mitigation. For example, if more suction is needed, the actuatable member 342 is further opened to increase flow. Further, any debris caught within the aspiration tubing 35 (through closure of the actuatable member 342) may be relieved as the actuatable member 342 is moved to a more open position. In some cases, the actuatable clamp 310 may prevent excess suction from being applied when the endoscope 200 is being removed from the patient P. For example, as the endoscope 200 is proximally retracted (withdrawn) from the patient P, the ILP may decrease. In response to the decrease in ILP pressure, the controller 30 may move the actuatable member 342 to a more closed configuration to reduce a flowrate or volume of outflow fluid.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid system, the system comprising:
a fluid management system including a fluid management console, comprising:
a housing;
a controller housed within the housing;
an inflow pump disposed within the housing and having a pump motor; and
a user input interface; and
a fluid cassette configured to be received within a receptacle of the housing of the fluid management console, the fluid cassette configured to provide a flow of fluid to a medical device;
a suction tubing operatively couplable with a suction source, the suction tubing configured to provide a flow of fluid from the medical device; and
an actuatable clamp positioned about an outer surface of the suction tubing.

2. The fluid system of claim 1, wherein the actuatable clamp is configured to selectively apply a compressing force to the suction tubing.

3. The fluid system of claim 1, wherein the actuatable clamp is movable between a fully open configuration and a fully closed configuration.

4. The fluid system of claim 3, wherein the actuatable clamp is positionable in a configuration between the fully open configuration and the fully closed configuration.

5. The fluid system of claim 3, wherein when the actuatable clamp is in the fully open configuration the flow of fluid from the medical device is at a maximum.

6. The fluid system of claim 3, wherein when the actuatable clamp is in the fully closed configuration the flow of fluid from the medical device is at a minimum.

7. The fluid system of claim 1, wherein the actuatable clamp is communicatively coupled to the controller of the fluid management console.

8. The fluid system of claim 1, wherein the actuatable clamp comprises at least one pressure sensor configured to measure a pressure within the suction tubing.

9. The fluid system of claim 1, wherein the actuatable clamp comprises an elongate tubular body having a lumen extending therethrough.

10. The fluid system of claim 1, wherein the actuatable clamp comprises a first body member hingedly coupled to a second body member.

11. The fluid system of claim 1, wherein the actuatable clamp comprises an actuatable member, the actuatable member configured to be displaced into a lumen of the actuatable clamp.

12. The fluid system of claim 11, wherein the actuatable member is coupled to an actuation member.

13. The fluid system of claim 1, wherein the actuatable clamp is configured to selectively reduce a diameter of the suction tubing.

14. The fluid system of claim 1, wherein the actuatable clamp is configured to apply a compressive force around less than an entirety of a circumference of the suction tubing.

15. The fluid system of claim 1, wherein the actuatable clamp is configured to apply a compressive force around an entirety of a circumference of the suction tubing.

16. A fluid system, the system comprising:
a fluid management system including a fluid management console, comprising:
a housing;
a controller housed within the housing;
an inflow pump disposed within the housing and having a pump motor; and
a user input interface; and
a fluid cassette configured to be received within a receptacle of the housing of the fluid management console, the fluid cassette configured to provide a flow of fluid to a medical device;
a suction tubing operatively couplable with a suction source, the suction tubing configured to provide a flow of fluid from the medical device; and
an actuatable clamp removably positioned about an outer surface of the suction tubing and in electronic communication with the controller of the fluid management console, the actuatable clamp configured to selectively change a volume of the flow of fluid through the suction tubing.

17. The fluid system of claim 16, wherein the actuatable clamp is movable between a fully open configuration and a fully closed configuration.

18. The fluid system of claim 17, wherein the actuatable clamp is positionable in a configuration anywhere between and including the fully open configuration and the fully closed configuration.

19. An actuatable clamp configured to selectively control a flow of fluid from a medical device, the actuatable clamp comprising:
an elongate tubular body extending from a proximal end to a distal end and having a lumen extending therethrough, the elongate tubular body comprising:
a first body member;
a second body member hingedly coupled to the first body member; and
an actuatable member configured to be selectively displaced into the lumen of the elongate tubular body;
wherein the first body member and the second body member are movable between a closed configuration and an open configuration configured to provide a lateral opening to the lumen of the elongate tubular body.

20. The actuatable clamp of claim 19, further comprising at least one pressure sensor disposed adjacent to the lumen of the elongate tubular body.
